(19) United States
(12) Patent Application Publication
(10) Pub. No.: US 2014/0206956 A1
(43) Pub. Date: Jul. 24, 2014

(54) DEVICE, SYSTEM AND METHOD FOR IN-VIVO IMMUNOASSAY

(71) Applicants: Elisha Rabinovitz, Haifa (IL); Zvika Gilad, Haifa (IL); Osnat Sella-Tavor, Kish (IL); Amit Pascal, Haifa (IL)

(72) Inventors: Elisha Rabinovitz, Haifa (IL); Zvika Gilad, Haifa (IL); Osnat Sella-Tavor, Kish (IL); Amit Pascal, Haifa (IL)

(21) Appl. No.: 14/237,019

(22) PCT Filed: Aug. 1, 2012
(86) PCT No.: PCT/IL12/50284

§ 371 Date: Feb. 21, 2014

Related U.S. Application Data
(60) Provisional application No. 61/515,196, filed on Aug. 4, 2011.

(51) Int. Cl. A61B 5/145; A61B 5/00; A61B 5/1459; A61B 5/1473; G01N 33/53; A61B 5/07

(52) U.S. Cl. CPC A61B 5/14503; G01N 33/5302; A61B 5/07; A61B 5/1459; A61B 5/1473; A61B 5/0084
USPC 600/302; 600/367; 600/310; 600/345; 600/476

(57) ABSTRACT

In vivo devices, systems and methods for in vivo immunoassay include inserting into a patient's body lumen an in vivo diagnostic device comprising a housing. The housing of the device comprises a chamber, and a chromatography strip for immunoassay of a body lumen substance. The housing may further comprise a casing for the chromatography strip. The casing may comprise a first opening to allow entrance of in vivo liquids into the casing and a second opening into the chamber. The housing may further comprise a sensor to sense a property of the chromatography strip. Following insertion of the device into the patient's body, a sample is collected and the immunoassay is performed in vivo in areas of pathological lesions for a predetermined period of time. An in vivo image may be acquired or other data such as colorimetric or intensity data may be obtained from the chromatography strip.

### FIELD OF THE INVENTION

The present invention relates to in vivo immunoassay in general, and to immunoassay using swallowable capsules in particular.

### BACKGROUND OF THE INVENTION

The basic principle of any immunochemical technique is that a specific antibody will combine with its specific antigen to give an exclusive antibody-antigen complex. Antigens are generally of high molecular weight and commonly are proteins or polysaccharides. Polypeptides, lipids, nucleic acids and many other materials can also function as antigens. Immune responses may also be generated against smaller substances, called haptens, if these are chemically coupled to a carrier protein or other synthetic matrices. A variety of molecules such as drugs, simple sugars, amino acids, small peptides, phospholipids, or triglycerides may function as haptens. Thus, given enough time, just about any foreign substance will be identified by the immune system and evoke specific antibody production.

Immunoassays are rapid, sensitive, and selective, and are generally cost effective. They have been applied to clinical diagnostics, environmental analysis and food safety assessment. Many types of immunoassay have been used to detect the presence of various substances, often generally called ligands, in body fluids such as blood and urine. These assays involve antigen antibody reactions, synthetic conjugates comprising radioactive, enzymatic, fluorescent, or visually observable metal sol tags, and specially designed reactor chambers. In these assays, there is a receptor, e.g., an antibody, which is specific for the selected ligand or antigen, and a means for detecting the presence, and often the amount, of the ligand-receptor reaction product. Most current tests are designed to make a quantitative determination, but in many cases all that is required is a positive/negative indication. For these tests, visually observable indicia such as the presence of agglutination or a color change are preferred.

The lateral flow immunoassay, which is also known as the immuno-chromatographic assay, or “strip” test, is an example of a widespread test that is simple to perform by almost anyone and operates more rapidly than traditional laboratory-based testing. This area of diagnostics has grown dramatically in recent years, with the most common and well-known of these being the home pregnancy test.

The principle of a lateral flow immunoassay relies on the competition for binding sites on polymer or metal particles. Antibodies that are raised to a specific target are bound to metal nanoparticles or dyed polymer particles. These particles are then applied using an immersion procedure onto a release pad in order to produce a stable particle reservoir for release onto a nitrocellulose-based membrane. Two lines of reagents are immobilized onto the membrane. The target reference or test line comprises a conjugate that can specifically bind the target to be identified and the other, the control, is a line of anti-species antibody. The release pad and membrane are assembled together with an absorbent pad. The sample is initially added to the adsorbent pad and then the strip is left for a few minutes with the result read directly by eye, looking for the presence of colored lines. These kits are relatively cheap to make. They also have a long shelf-life and are fully disposable. This technology is, therefore, ideally suited to any rapid diagnostics.

Most medical detection kits utilizing the lateral flow immunoassay are based on in vitro testing of body fluid, such as urine or blood. For example, in some cases, diseases, such as cancer, are detected by analyzing the blood stream for tumor specific markers, typically, specific antibodies.

Another example is the presence of elevated concentrations of red blood cells in the gastrointestinal (GI) tract that may indicate different pathologies, depending on the location of the bleeding along the GI tract. Thus, for instance, bleeding in the stomach may indicate an ulcer, whereas bleeding in the small intestine may indicate the presence of a tumor. Furthermore, different organs may contain different body fluids requiring different analysis methods. For example, the stomach secretes acids, whereas pancreatic juice is basic.

Thus, early in vivo detection, identification and location of abnormal conditions (such as, for example, an atypical presence or concentration of a substance in body fluids) may be critical for definitive diagnosis and/or treating of various pathologies.

It is, therefore, an object of the present invention to provide a rapid, sensitive method for in vivo detection of low levels of various ligands, antigens or antibodies in body fluids, which involves a minimal number of procedural steps and yields reliable results. Another object is to provide an in vivo immunoassay which has high sensitivity and fewer false positives than conventional assays. Yet another object is to provide an in vivo diagnostic device and immunoassay system for in vivo detection of low levels of ligands, antigens or antibodies in body fluids.

### Overview of the Lateral Flow Immunoassay

As an introductory part of the invention, reference is initially made to FIG. 1 showing a perspective view of an exemplary chromatography strip. Such strip is widely used in the lateral flow immunoassay. It should be noted that FIG. 1 relates to prior art knowledge, and as such it merely constitutes a reference for better understanding of the present invention.

The typical chromatography strip shown on FIG. 1, used in the lateral flow immunoassay, consists of the following components:
Sample (absorbent) pad 11, onto which the body lumen liquid sample may be applied;
Conjugate (reagent) pad 12, which may contain antibodies specific to the target analyte molecules (ligands or antigens) and which may be conjugated to colored particles, such as colloidal metal (e.g., gold) particles or polymer (e.g., latex) microspheres;
Reaction membrane 13, e.g., a hydrophobic nitrocellulose or cellulose acetate membrane, onto which anti-target analyte antibodies are immobilized in a line across the membrane as a test line 16 and a control line 17 (a control line may contain either antigens or antibodies specific for the conjugate antibodies); and
Wick or waste pad (reservoir) 14, which is a further absorbent pad designed to draw the sample across the reaction membrane by capillary action and collect it.

The above listed components of the chromatography strip may be fixed to an inert backing material 15, such as plastic backing.

The chromatography strip may be made of, for example, a pad or other support structure, such as a flat piece or a unit of another shape, coated with or impregnated or otherwise including nitrocellulose or any other polymer suitable for a chromatographic process. The strip may be in a form of a plain narrow piece (i.e., “strip-shaped”), or may not necessarily be strip-shaped (e.g., if the flow is capillary creeping flow). It may, for example, be coil-shaped in order to increase its length in the same volume, and hence, improve separation of the components of the body fluid.

Essential in the lateral flow immunoassay is the movement of a liquid sample, or its extract containing the analyte of interest, along the chromatography strip thereby passing various zones of the strip where binding molecules have been attached that exert more or less specific interactions with the analyte.

At one end of chromatography strip, sample (absorbent) pad 11 is provided. The sample pad 11 is usually made of cellulose, glass fiber, cross-linked silica or other material where the body fluid sample is initially drawn from the exterior body lumen and then subjected to the lateral flow immunoassay. If necessary, sample pad 11 may optionally modify the sample to improve the results of the assay. This might be by modifying pH, filtering out solid components, separating whole body fluid constituents, adsorbing out unwanted particles and compounds or some other test specific variable. For some applications, the sample pad may be pre-treated by being dipped into a specific buffer containing a mix of a solution comprised of soluble proteins, surfactants, detergents and other polymers. Such buffer allows for a steady lateral flow and prevents nonspecific binding of sample components to the pad.

In close contact with plastic backing 15 and sample pad 11 is conjugate (reagent) pad 12, which is usually made of cross-linked silica. A colored reagent, such as a detection labeled conjugate, is dried down and held in place on this pad.

After absorbing the drawn body liquid onto sample pad 11, the liquid moves into conjugate pad 12 by capillary action, re-hydrates the labeled conjugate particles and allows the mixing of these particles with the absorbed body liquid. The labeled conjugate interacts with the specific analyte contained in the drawn body liquid flow, thereby initiating the intermolecular interactions, which are dependent on the affinity and avidity of the reagents. These interactions will continue during the entire chromatographic separation process.

The labels may be prepared of colored or fluorescent nanoparticles for optical detection. In principle, any colored particles can be used. However, commonly either latex (blue color) or nanometer sized particles of gold (red color) are used. The gold particles are red in color due to localized surface plasmon resonance. Fluorescent or magnetic labeled particles can also be used; however, these require the use of an electronic reader to assess the test result.

The labels are normally of the sizes of 0.01 mm to 1 mm, allowing an unobstructed flow through the membrane. The labels may be selenium particles, carbon macrocycles or liposomes, besides the aforementioned colloidal gold and latex particles. In colored liposomes, fluorescent or bioluminescent dyes can be incorporated, allowing visualization, and, when applicable, quantification of the response. The newest labels may also include quantum dots.

As mentioned above, conjugate pad 12 is usually made of cross-linked silica, but it may also be made from non-absorbent material such as fiberglass, polyester, rayon or any other similar material. The conjugate pad is preferably comprised of a synthetic material (at least when using a gold conjugate) to ensure the efficient release of its contents. Pre-treatment of the conjugate pad helps to ensure that the conjugate releases at the proper rate and enhances its stability. The pre-treatment is performed in the same way as with the sample pad.

The complex of the labeled conjugate and analyte then moves into reaction membrane 13. Membrane 13 may be produced from nitrocellulose, nylon, polyethersulfone, polyethylene or fused silica.

If produced from nitrocellulose, membrane 13 consists of a very thin Mylar sheet coated with a layer of nitrocellulose (NC). The benefits of using NC as an immunoassay matrix include low cost, good capillary flow, high binding affinity for protein, ease of handling and cutting, as well as the ability of manufactures to varying thickness and components of the membrane in order to suit the specific application. The NC membrane binds proteins electrostatically through an interaction with the nitrate esters and the peptide bonds of the protein.

As shown on FIG. 1, at least two lines are sprayed on the strip: a test line 16 and a control line 17, which have both been pre-treated with specific antibodies or antigens (ligands), and which is the standard for lateral flow immunoassays. These lines are usually closer to wicking pad 14 than to conjugate pad 12 in order to improve the overall performance of the lateral flow immunoassay. Some lateral flow assays may have more than one test line, but each additional test line greatly increases the complexity of development, and thus increases cost.

Initially, the complex of the labeled conjugate and analyte moves onto membrane 13. Then it starts migrating towards test line 16 capturing and recognizing the binding analyte, where it becomes immobilized and produces a distinct signal for example, in the form of a colored line, indicating the test is complete. A distinct signal at control line 17 may indicate a proper flow of the body liquid through chromatography strip 3. Depending upon the analytes present in the body liquid and on the type of the immunoassay performed, the colored reagent can become bound at test line 16 and at control line 17, or, alternatively, only at test line 16.

The so called “wick” (wicking or waste) pad 14 maintains a lateral flow along the chromatography strip. Wick pad 14 may be made of non-woven, cellulose fiber sheets. These pads can be manufactured in a variety of thicknesses and densities to suit the needs of the immunoassay.

There are different types of lateral flow immunoassays available on the market. For example, in the double antibody sandwich immunoassay, the drawn body fluid migrates from sample pad 11 through conjugate pad 12 where any target analyte present will bind to the labeled conjugate particles. The sample fluid mixture then continues to migrate across the membrane until it reaches test line 16, where the target/conjugate complex binds to the immobilized antibodies, producing a visible line on membrane 13. The fluid then migrates further along the strip until it reaches control line 17, where excess conjugate binds and produces a second visible line on the membrane. Control line 17 is therefore indicative of the sample that has migrated across membrane 13 as intended. Thus, two colored lines 16 and 17 appearing on membrane 13 is a positive result. A single colored control line 17 is a negative result. Double antibody sandwich assays are most suitable for larger analytes, such as bacterial pathogens and viruses, with multiple antigenic sites.

Competitive assays are primarily used for testing small molecules and differ from the double antibody sandwich immunoassay in that the conjugate pad contains antibodies that are already bound to the target analyte or to an analogue thereof. If the target analyte is present in the sample, it will therefore not bind with the conjugate and will remain unlabeled. As the sample migrates along reaction membrane 13 and reaches test line 16, an excess of unlabeled analyte will bind to the immobilized antibodies and block the capture of the conjugate, so that no visible line is produced. The unbound conjugate will then bind to the antibodies in control line 17, producing a colored line. The single colored control line 17 on reaction membrane 13 is a positive result. Two colored lines 16 and 17 is a negative result. However, if an excess of unlabeled target analyte is not present, a weak line may be produced in test line 16, indicating an inconclusive result. Competitive assays are most suitable for testing of small molecules, such as mycotoxins, unable to bind to more than one antibody simultaneously.

There are a number of variations on lateral flow immunoassay technology. Test line 16 on membrane 13 may contain immobilized antigens or enzymes (depending on the target analyte) rather than antibodies. In this case, as above, two colored lines 16 and 17 indicate a negative result, whereas one single colored control line shows a positive result. In a slightly modified format, the competitive immunoassay may be also used for detection of specific antibodies in the body fluid. It is also possible to apply multiple test lines to create a multiplex immunoassay.

Lateral flow immunoassays are simple to use by untrained operators and generally produce a result within several minutes. Lines 16 and 17 can take as little as a few minutes to develop. Generally, there is a tradeoff between time and sensitivity, such that more sensitive tests may take longer to develop. The lateral flow immunoassays typically require little or no sample or reagent preparation. They are very stable and robust, have a long shelf life and do not usually require refrigeration. They are also relatively inexpensive to produce. These features make them ideal for use in the in vivo diagnostic device according to the embodiments of the invention.

### SUMMARY OF THE INVENTION

Various embodiments of the invention provide devices, systems and methods for in vivo immunoassay including the in vivo detection of a preselected ligand, antibody or antigen in a liquid sample such as body fluid.

In one embodiment, the in vivo diagnostic device may be an autonomous swallowable capsule. The device may be assembled inside a case (shell or housing), which may optionally have a substantially transparent portion.

In another embodiment, an in vivo diagnostic device may include a chromatography strip for in vivo immunoassay of a body lumen substance and a sensor to sense in vivo a property (e.g., color, color intensity, color change, radiation, emitted signal, emitted radiation, change of a characteristic, or the like) of the chromatography strip. The sensor may be, for example, a photodiode, a photodiodes array, an electrochemical sensing unit, a magnetic field sensing unit, an imager, an image sensor, a light detector, a color detector, a light-sensitive unit, a color-sensitive unit, or the like.

In a further embodiment, the chromatography strip is generally internal to the case of the device. The chromatography strip is inserted into a tube, a sleeve, a pipe, or other casing for the strip (hereinafter, the “tube”). This tube is located inside the case of the device and insulates the chromatography strip from the interior of the device.

In a particular embodiment, the case of the device may comprise a chamber, which is isolated from the interior of the device with the isolating element, such as plug or isolating ring, and which may optionally be transparent.

In another embodiment, the tube has one end opening inside the chamber of the device and a second end, which may be opened to the outside of the device's housing. There is an ambient atmospheric pressure inside the chamber of the device and consequently, inside the tube, which enables the capillary forces to draw the body lumen liquid along the chromatography strip.

In yet another embodiment, the in vivo diagnostic device may include a sealing element or gate or plug to seal the second end of the tube, covering the entrance of the tube. The body lumen liquid may therefore enter the device only via the chromatography strip located inside the tube. In some embodiments, the sealing element or gate or plug may be openable, for example, only if a pre-defined condition is met, at a specific time, at a specific location, or the like. In a particular embodiment, at least a portion of the chromatography strip may be covered by a dissolvable plug.

In a further embodiment, the sensor for sensing an in vivo property of the chromatography strip may reside on a base or any other suitable support, as described in PCT Application Publication No. WO 2006/003649, assigned to the common assignee of the present invention. The base may be a stepped printed circuit board (PCB). The base may optionally include one or more components, e.g., conductive rings, and/or conductive step. Other designs, components, elements, and structures may be used in addition to and/or in place of the rings, steps, etc. The PCB may optionally include other components of the device such as a sensor for sensing the current location of the device and antenna typically associated with a transmitter for transmitting data from the device to an external system. The PCB may further include contact points to connect additional components.

In yet a further embodiment, the device may include an imager in order to acquire an in vivo image of the chromatography strip through the housing portion and a power source, such as batteries. In some embodiments, the in vivo device may additionally include an in vivo camera to acquire an in vivo image of a body lumen.

In still a further embodiment, a system of the invention may include the in vivo diagnostic device, an external receiver/recorder able to receive data (e.g., image data) transmitted by the in vivo device, and a computing platform or workstation able to store, process, display, or analyze the received data.

Some embodiments of the invention may include a method of in vivo diagnostics based on the performed immunoassay. The method may include the following steps:
inserting into a patient's body lumen an in vivo diagnostic device of the present invention;
collecting the sample and performing the immunoassay in vivo in the areas of the pathological lesions for the predetermined period of time; and
acquiring an in vivo image or obtaining other data such as colorimetric or intensity data, of a chromatography strip.

The method may further optionally include acquiring in vivo an image of the body lumen; transmitting the acquired in vivo image or other data of the chromatography strip; analyzing the in vivo image or data of the chromatography strip; and/or other suitable operations.

Various embodiments of the invention may allow various benefits, and may be used in conjunction with various applications. The details of one or more embodiments are set forth in the accompanying figures and the description below. Other features, objects and advantages of the described techniques will be apparent from the description and drawings and from the claims.

### BRIEF DESCRIPTION OF THE DRAWINGS

The present invention will be understood and appreciated more fully from the following detailed description taken in conjunction with the appended figures. Various exemplary embodiments are well illustrated in the accompanying figures with the intent that these examples not be restrictive. It will be appreciated that for simplicity and clarity of the illustration, elements shown in the figures referenced below are not necessarily drawn to scale. Also, where considered appropriate, reference numerals may be repeated among the figures to indicate like, corresponding or analogous elements. Of the accompanying figures:

FIG. 1 is a schematic side view of a typical prior art chromatography strip used in lateral flow immunoassays;

FIG. 2A is a cross-sectional side view of an in vivo diagnostic device, constructed and operative in accordance with an embodiment of the present invention;

FIG. 2B is a cross-sectional front view of an in vivo diagnostic device, constructed and operative in accordance with an embodiment of the present invention;

FIG. 2C is a cross-sectional back view of an in vivo diagnostic device, constructed and operative in accordance with an embodiment of the present invention;

FIG. 3 is a cross-sectional view of an in vivo diagnostic device having an air locked compartment filled with foaming material inside the chamber;

FIG. 4A is a schematic view of a single photodiode sensor setup in accordance with an embodiment of the present invention;

FIG. 4B is a schematic view of a sensor setup including several pairs of LEDs and photodiodes for simultaneous testing of different proteins;

FIG. 4C is a schematic view of a sensor setup including a photodiode array and a single back LED;

FIG. 5 is a schematic illustration of a system for obtaining colorimetric or intensity data, or the like, from the in vivo diagnostic device in accordance with one embodiment of the present invention; and

FIGS. 6A-C are flow charts describing methods of using an in vivo diagnostic device in accordance with embodiments of the present invention.

It will be appreciated that for simplicity and clarity of illustration, elements shown in the figures have not necessarily been drawn to scale. For example, the dimensions of some of the elements may be exaggerated relative to other elements for clarity. Furthermore, where considered appropriate, reference numerals may be repeated among the figures to indicate corresponding or analogous elements.

### DETAILED DESCRIPTION OF THE INVENTION

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

It should be noted that although a portion of the discussion may relate to in vivo diagnostic devices, systems, and methods, the present invention is not limited in this regard, and embodiments of the present invention may be used in conjunction with various other in vivo sensing and imaging devices, systems, and methods. As such, some embodiments of the invention may be used, for example, in conjunction with in vivo sensing of pH, temperature, pressure and/or electrical impedance, in vivo detection of a substance or a material, in vivo detection and imaging of a medical condition or a pathology, in vivo acquisition or analysis of data, and/or various other in vivo sensing and imaging devices, systems, and methods. Some embodiments of the invention may be used not necessarily in the context of in vivo imaging or in vivo sensing.

The in vivo diagnostic device of an embodiment of the invention may typically be fully autonomous and typically self-contained. For example, a device according to some embodiments may be a capsule or other unit where all the components are substantially contained within a case, housing or shell, and where the device does not require wires or cables in order to receive power or transmit information, for example.

According to a particular embodiment of the invention, the in vivo device is essentially floatable or has a neutral or near neutral buoyancy in water or in other liquids that may fill body lumens. Accordingly, the device may have a specific gravity of 1 in reference to water or it may have a specific gravity of about 1 in reference to water. The in vivo device according to an embodiment may be designed to access pathologic lesions in nearly every region of the gastrointestinal (GI) tract, including the colon and biliary tree. In some embodiments, the in vivo device may be designed to collect the samples in the pathological areas only and to bypass the healthy areas, and it may be designed to access difficult to reach areas, where tethered endoscopes cannot reach or cannot reach easily.

Some embodiments of the present invention are directed to a typically swallowable in vivo diagnostic device in a form of a swallowable capsule that may be used for diagnosing the pathological areas inside the GI tract.

In general, devices according to embodiments of the present invention may be similar to devices described in the following publications, all of which are assigned to the common assignee of the present invention:
U.S. Pat. No. 7,009,634 entitled “Device and System for In-vivo Imaging”,
U.S. Pat. No. 5,604,531 entitled “In-Vivo Video Camera System”, and/or
U.S. application Ser. No. 11/597,245 entitled “Device, System and Method for In-Vivo Analysis”.

In addition, components of the device according to embodiments of the present invention may be similar to components used in the PillCam® capsule endoscopy system commercially available from the common assignee of the present invention. Of course, devices, systems, structures, functionalities and methods as described herein may have other configurations, sets of components and processes, etc.

It should be also noted that while a device, system and method in accordance with some embodiments of the invention may be used, for example, in a human body, the invention is not limited in this respect. For example, some embodiments of the invention may be used in conjunction with or inserted into a non-human body, e.g., a dog, a cat, a rat, a cow, or other animals, pets, laboratory animals, etc.

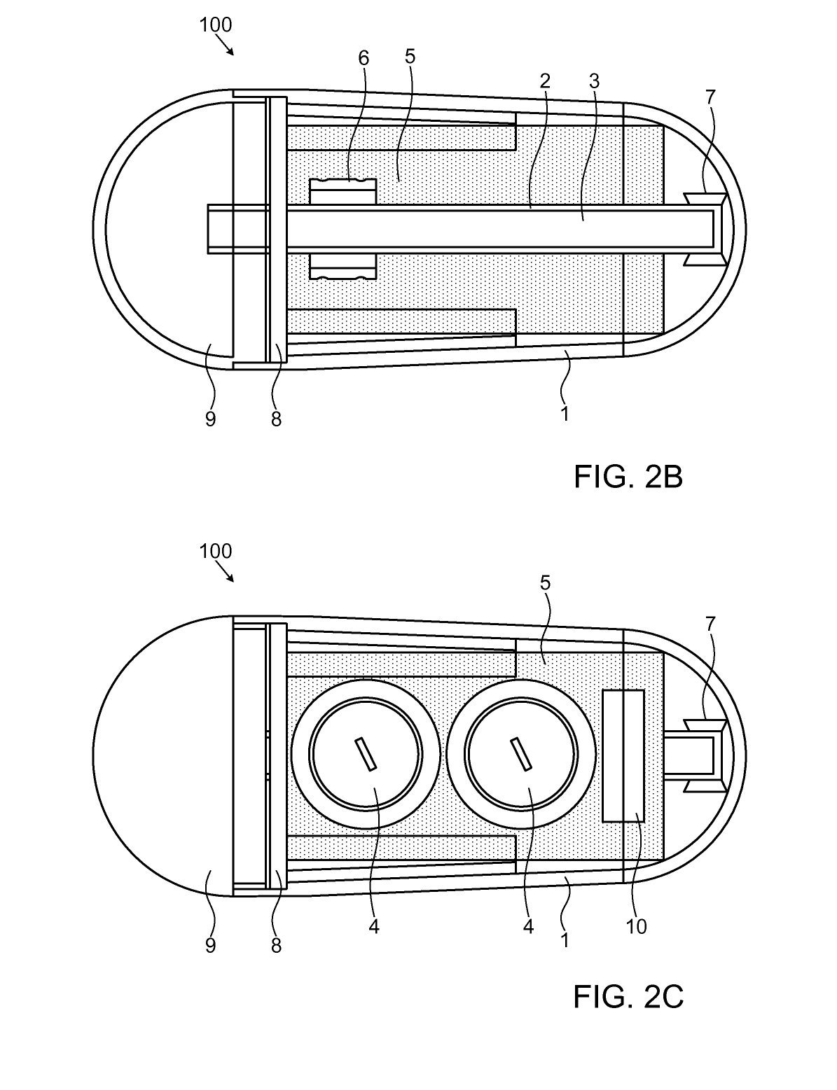

Reference is now made to FIGS. 2A, 2B and 2C, which depict an in vivo diagnostic device according to embodiments of the invention, for example, an autonomous swallowable capsule. Device 100 may be ovoid or capsule shaped such that it can be easily swallowed and moved through the GI tract by natural peristalsis, or guided through the GI tract or blood vessels using external force, e.g., magnetic fields.

As shown in FIGS. 2A-2C, all the components of device 100 are substantially contained within housing 1, which may be transparent or not. The case may have the following dimensions: 5-12 mm in width and 10-32 mm in length, though other dimensions are possible.

Device 100 may include at least one chromatography strip 3, which is internal to the case of device 100. The walls of housing 1 may be made of any suitable biocompatible polymeric material, such as polycarbonate, polystyrene, parylene, parylene C and isoplast, and may taper as shown in FIGS. 2A-2C or it may be substantially parallel or it may have any other suitable shape.

The chromatography strip 3 is enclosed within optically transparent tube 2, which is located inside the case of device 100 and isolates the strip from the interior of device 100 and from endoluminal fluids.

Tube 2 or portions thereof may be made of any transparent material, for example, glass or any typically biocompatible polymers, such as parylene, parylene C and isoplast, and may be in a form of a sleeve, pipe, coil or any other casing for strip 3, which may not necessarily be strip-shaped. Chromatography strip 3 may be a commercially available strip, for example the Helicobacter pylori stool antigen (HP Ag) test strip manufactured by JD Biotech for detection of H. pylori antigen. Other proteins inside the GI tract, for example calprotectin, lactoferrin, carcinoembryonic antigen (CEA), cancer antigen 19-9 (CA19-9), pancreatic elastase and amylase, may be in vivo detected using the corresponding chromatography strips.

As noted above, strip 3 may not necessarily be in a form of a plain narrow piece (i.e., “strip-shaped”). It may be coil-shaped, for example, in order to increase its length, and hence, improves separation of the components of the body fluid. The coil-shaped strip may be useful in detection of several different proteins within the same in vivo diagnostic device, as will be explained below.

Device 100 may further comprise chamber 9, which is isolated from the interior of device 100 by isolating element 8, such as plug or isolating ring. Chamber 9 may be dome-shaped and positioned at one end of device 100, or it may have any other shape and position. The volume of the chamber may be 600-700 μL.

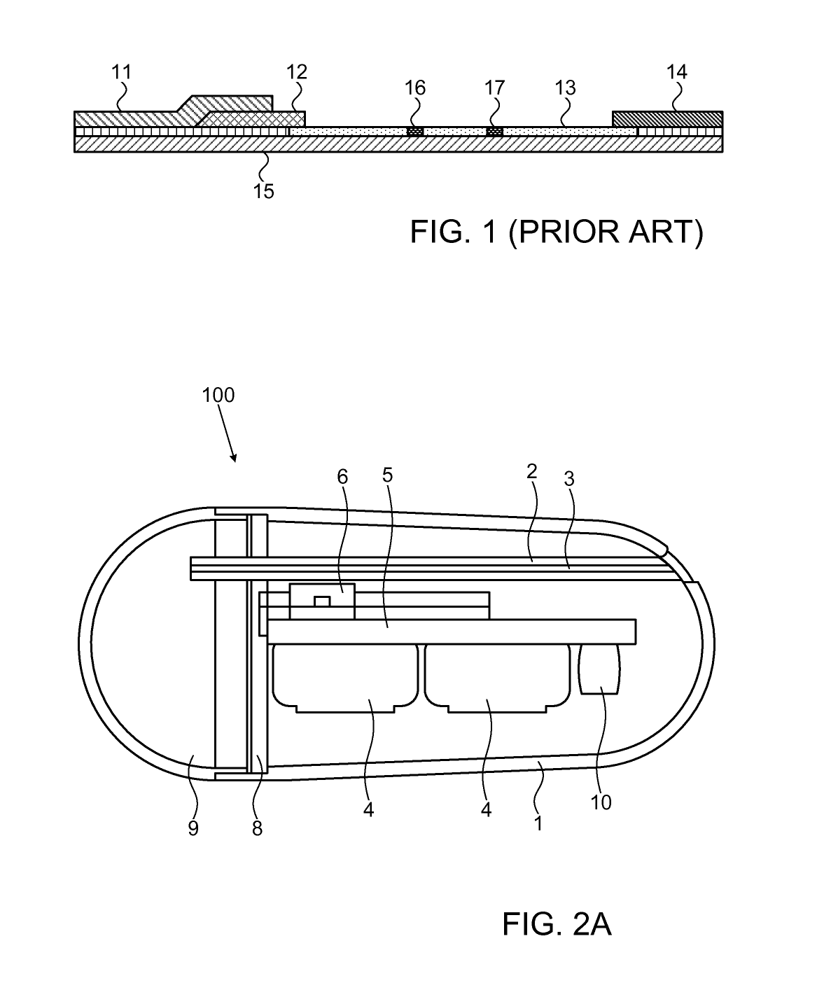

According to a particular embodiment, tube 2 has one end opening inside chamber 9 of device 100. This is done in order to ensure normal atmospheric pressure inside tube 2 as a result of the same ambient pressure inside chamber 9. Wick pad 14 of chromatography strip 3 (shown in FIG. 1) extends into chamber 9 and pulls fluid off reaction membrane 13 to allow the capillary flow along strip 3 to keep flowing in the proper direction and at the proper rate. Consequently, liquids drawn from exterior body lumens into tube 2 may easily move along chromatography strip 3 under capillary action. Wick pad 14 and preservation thereof under normal atmospheric pressure of chamber 9 is to ensure that the body liquid does not back flow down the membrane and raise the background or possibly cause false positives.

Alternatively, a slightly positive pressure inside tube 2 compared to chamber 9 may be created by difference in temperatures of an interior, exterior of device 100 and of chamber 9, as explained below.

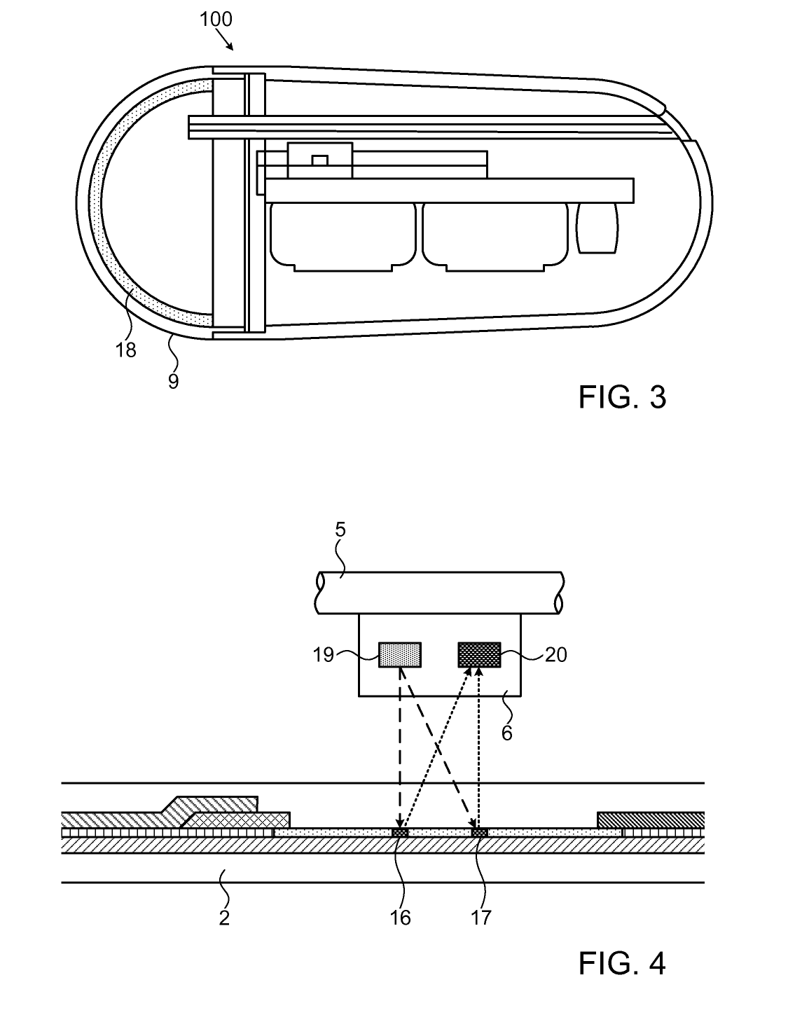

Reference is now made to FIG. 3 showing a cross-sectional view of the in vivo device in accordance with an embodiment of the invention, having an air locked compartment 18 inside chamber 9. The compartment may be filled with any thermal insulating material, such as polyurethane, polyimide or polystyrene foam. The inner wall of compartment 18 may be made of any suitable biocompatible polymeric material, such as polycarbonate, polystyrene, parylene, parylene C and isoplast. Other insulation methods may be used.

In this embodiment of this invention, device 100 may initially be stored in a refrigerator or may otherwise be cooled to a temperature of approximately 5-10° C. and swallowed relatively cold by a patient. The temperature of the interior of device 100 and of tube 2 containing strip 3 rapidly increases after swallowing and eventually reaches the temperature of the patient's body. The temperature of chamber 9 is however increased slower than the temperature of tube 2 and of the rest interior of device 100 because chamber 9 is insulated by insulating compartment 18 from one side and isolating element 8 from another side. As the temperature gradient between tube 2 and chamber 9 is created, the air pressure gradient from tube 2 towards chamber 9 is also created. This gradient stimulates the capillary action on chromatography strip 3.

As shown in FIGS. 2A and 2B, the in vivo diagnostic device of the invention may include sealing element 7 in a form of, e.g., a gate or plug to seal the second end of tube 2.

Sealing element 7 may be positioned at one end of tube 2 covering the entrance of the tube. The body lumen liquid may therefore enter device 100 only via chromatography strip 3 located inside tube 2. In some embodiments, sealing element 7 may be openable, for example, only if a pre-defined condition is met, at a specific time, at a specific location, or the like.

In a particular embodiment, at least a portion of tube 2 may be covered, for example, by sealing element 7. In some embodiments, sealing element 7 may be a dissolvable plug, which may be exposed to external body fluids. Plug 7 may comprise a layer or a plurality of layers of impermeable or slightly permeable material or a combination of materials that is essentially durable (i.e., does not corrode or disintegrate) under in vivo conditions. Thus, plug 7 may serve to seal tube 2 of in vivo device 100 from the body lumen fluids, while it is intact. In addition, plug 7 may serve as a gate, and may be made of biodegradable or other disintegrating materials, such as carbohydrates, gelatine, wax, or the like.

According to some embodiments, plug 7 may be disintegrated or otherwise perforated, such that chromatography strip 3 can come into contact with environmental fluids (e.g., body lumen fluids), and a fluid sample may be drawn into strip 3. Plug 7 may be disintegrated according to suitable methods, for example, in a time-dependent manner (e.g., according to the width or other, typically mechanical properties of plug 7), in a pH dependent manner, due to a specific enzymatic environment or specific prevailing bacteria or other fauna, in a temperature dependent manner, depending on a prevailing electromagnetic field, or the like. A specific example of material that would result in opening of plug 7 in the stomach (or cecum) under enzymatic action of bacteria is gelatine.

In a particular embodiment, plug 7 is made of a polymethacrylate polymer, such as Eudragit®, which dissolves at rising pH values, for example, when in vivo diagnostic device 100 reaches the pylorus. The different grades of commercially available Eudragit® can be combined with each other, making it possible to adjust the dissolution pH, and thus to achieve the required GI targeting for conducting the test.

An example of material that would result in opening of plug 7 in a time-dependent manner is Parylene® C, which is a coated hydrogel polymer, such as ethyl cellulose acetate. Parylene® C, which is a dimer of poly-p-xylene with a substitution of a single chlorine molecule, provides a combination of properties such as a low permeability to moisture, chemicals, and other corrosive gases. For example, in an in vivo diagnostic device having a diameter of 11 mm, plug 7 may be made of a layer of Parylene C having a thickness of between 5 to 20 μm. In another embodiment of the invention, plug 7 may be made of a 10 μm thick layer of Parylene C and a 0.5 mm thick layer of gelatine. The gelatine, which may be soft, hard or vegetable gelatine, may be cross-linked to increase its durability.

In one embodiment of the invention, in vivo diagnostic device 100 may include sensor 6 to sense in vivo a property (e.g., color, color intensity, color change, radiation, emitted signal, emitted radiation, change of a characteristic, or the like) of chromatography strip 3. Sensor 6 may be a typical white (narrow-band) analogue sensor or digital sensor that integrates the analogue to digital convertor (ATD) to output the signal proportional to voltage. Specific examples of sensor 6 may be a photodiode, a photodiodes array, a spectrophotometer, a fluorometer, a light or color detector, light-sensitive or color-sensitive unit, an electrochemical sensor or a magnetic field sensor.

Sensor 6 for sensing an in vivo property of the chromatography strip may reside on base 5 or any other suitable support, such as a stepped substrate as described in PCT Publication WO 2006/003649, assigned to the common assignees of the present invention. The base may be a stepped printed circuit board (PCB) or other suitable structure. Base 5 may optionally include one or more components, for example, conductive rings, and/or conductive step (not shown). Other designs, components, elements, and structures may be used in addition to and/or in place of the rings, steps, etc. The PCB may optionally include other components of device 100 such as a sensor for sensing the current location of the device and antenna typically associated with a transmitter for transmitting data from the device to an external system. The PCB may further include contact points to connect additional components.

Other components of in vivo diagnostic device 100 may be mounted on or incorporated in base 5, such as a sensor (not shown) for sensing the current location of the in vivo device, a radio frequency identification (RFID) tag, and an antenna typically associated with transmitter 10 for wirelessly transmitting data from the device to an external receiver. Base 5 may include contact points to connect additional components.

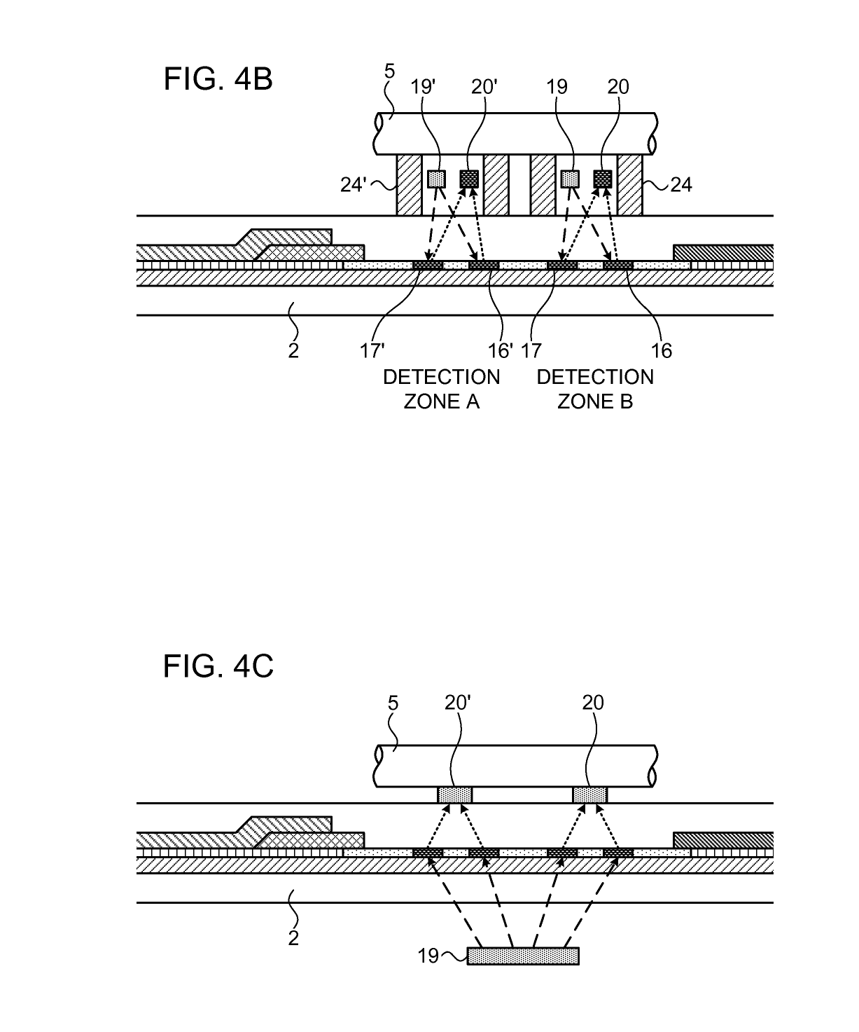

Reference is now made to FIG. 4A showing a single photodiode sensor setup for detecting colored lines 16 and 17 developing on chromatography strip 3 located inside tube 2.

According to an embodiment of the invention, sensor 6 attached to base 5 may comprise a monochrome light source, e.g., monochrome light-emitting diode (LED) 19, typically near-infrared or red, and a single photodiode 20, which is pre-calibrated for the relative voltage as a function of the reflected light intensity, in order to obtain quantitative results.

If two colored lines 16 and 17 appear on strip 3, the reflected light detected by photodiode 20 is of the highest possible intensity that results in the relatively high voltage signal on photodiode 20. In the most typical immunoassay described above, this is the indication of a positive test with respect to a particular analyte. On the other hand, if the signal from the photodiode 20 is absent, it means that none of the colored lines 16 and 17 has developed, and that the test is false. The relatively low voltage signal (by calibration) indicates that there is either one line developed and the test is negative, or the test is false. The test may then be repeated to confirm the negative result.

According to some embodiments, LED 19 may be a white LED lighting up lines 16 and 17 with white light. In this case, instead of the single photodiode 20, sensor 6 may comprise three photodiodes (R, G, B) and their respective filters.

According to some embodiments, device 100 may include multiple tubes 2 each housing a strip, wherein each tube may be sealed by a plug 7 having different characteristics, such that each plug 7 may be opened or disintegrated at a different location along the passage of device 100 through the GI tract.

In one embodiment, device 100 may include more than one (multiple) chromatography strips 3, which may be for testing the same or different proteins. In such case, multiple associated tubes 2 and other related multiple components of device 100 may be used, for example, for obtaining duplicates, for sampling a body lumen at more than one time point and/or in more than one location along the body lumen.

In another embodiment, when the in vivo testing of different proteins is required, instead of multiplication of strips 3 and other related components of device 100, one chromatography strip 3 having several detection zones (lines 16 and 17 in FIG. 1) may be used. In this case, it is a function of the optical design of the in vivo diagnostic device whether multiple sensors (for each control and test line) are used or only one sensor capable of simultaneous detection of different wavelengths signals from different detection zones is used.

Reference is now made to FIGS. 4B and 4C showing two sensor setups for simultaneous in vivo detection of two different proteins. FIG. 4B schematically shows a sensor setup including several pairs of LEDs and their corresponding photodiodes. In this setup, each pair of LEDs and photodiodes occupies a separate compartment in order to ensure only one proper detection zone is lit up with each specific LED, and to receive the signal from this particular zone to only one respective photodiode. For example, as shown in FIG. 4B, there are two detection zones A and B on the chromatography strip for detection of two different proteins, respectively, i.e., two pairs of test lines (16 and 16′) and two pairs of control lines (17 and 17′). Each detection zone is lit up with its respective LED (19 or 19′) and sensed with the corresponding photodiode (20 or 20′). Separate compartments 24 and 24′ optically isolate the pairs of LED-photodiode (19-20 and 19′-20′) from each other. In addition, different optical filters may be used for each specific white LED to provide different wavelength illumination for each detection zone.

Reference is now made to FIG. 4C, which shows a schematic view of a sensor setup comprising two detection zones, a photodiode array and a LED. The photodiodes 20 and 20′ attached to base 5 form an array of photodiodes, which is adjacent to the detection zones (lines) on the chromatography strip.

As shown in FIG. 4C, the photodiodes 20 and 20′ may be positioned very close to tube 2, just opposite to the detection zones. This may be done in order that each photodiode would sense only one respective zone on the chromatography strip. A single LED 19 may lights the strip from the side of tube 2, which is opposite the side of tube 2 where photodiodes 20 and 20′ are positioned.

Sensor 6 may be, but need not necessarily be, capable of acquiring an image of the chromatography strip or a portion thereof. In such case, the sensor may be selected from the group of the following cameras: complementary metal oxide semiconductor (CMOS) camera or charge coupled device (CCD) camera, an image sensor, a digital camera, a stills camera or video camera, or other suitable imagers, cameras, or image acquisition components.

In a particular embodiment of the invention, sensor 6 may be used to detect the appearance of lines 16 and 17 on chromatography strip 3 based on a regular barcode scanner. Such “barcode scanner” sensor may comprise one monochrome LED in combination with a low resolution camera to image the location of the test and control lines along the chromatography strip and hence, visually estimate the results of the test. There are numerous examples of miniaturized barcode scanners, which are commercially available, such as JADAK® barcode scanner IT5000 powered by ADAPTUS™ imaging technology 5.0, SICK OPTIC® miniature barcode reader CLV420, PSC® miniature barcode scanner LM520, and MARSON® miniature barcode scanners of the MT series.

The components of in vivo diagnostic device 100 may receive power from a power source 4, which may take the form of internal batteries, power cells, or power circuitry such as a wireless power receiving unit based on RF power transmission, which may be included in device 100. The battery within power source 4 may be very small. An example of a suitable battery is a silver oxide battery often used to power watches, lithium batteries or any other suitable electrochemical cells having a high energy density. For example, the battery may have a voltage of 1.55 volts and a capacity of 12.5 mA-hours and may be of a disk-like shape with a diameter of approximately 5.7 mm and a thickness of approximately 1.65 mm. With a typical range of power requirements, the battery may be expected to power in vivo device 100 for between approximately two weeks to eighteen months, depending on actual usage conditions. Other suitable power sources may be used. For example, power source 4 may receive power or energy from an external power source (e.g., an electromagnetic field generator), which may be used to transmit power or energy to in-vivo diagnostic device 100.

In further embodiments, battery power source 4 may be rechargeable via induction or ultrasonic energy transmission, and includes an appropriate circuit for recovering transcutaneously received energy. For example, battery power source 4 may include a secondary coil and a rectifier circuit for inductive energy transfer. In still other embodiments, battery power source 4 may not include any storage element, and in vivo device 100 may be fully powered via transcutaneous inductive energy transfer. As an example, such battery power source is commercially available from Medtronic, Inc. of Minneapolis, Minn.

In some embodiments, data collected or sensed by in vivo device 100 (e.g., images and image data taken from strip 3 or from the body lumen) may be transmitted by transmitter 10 to an external receiver or recorder unit, which may be portable, non-portable, mobile, non-mobile, wearable, or the like.

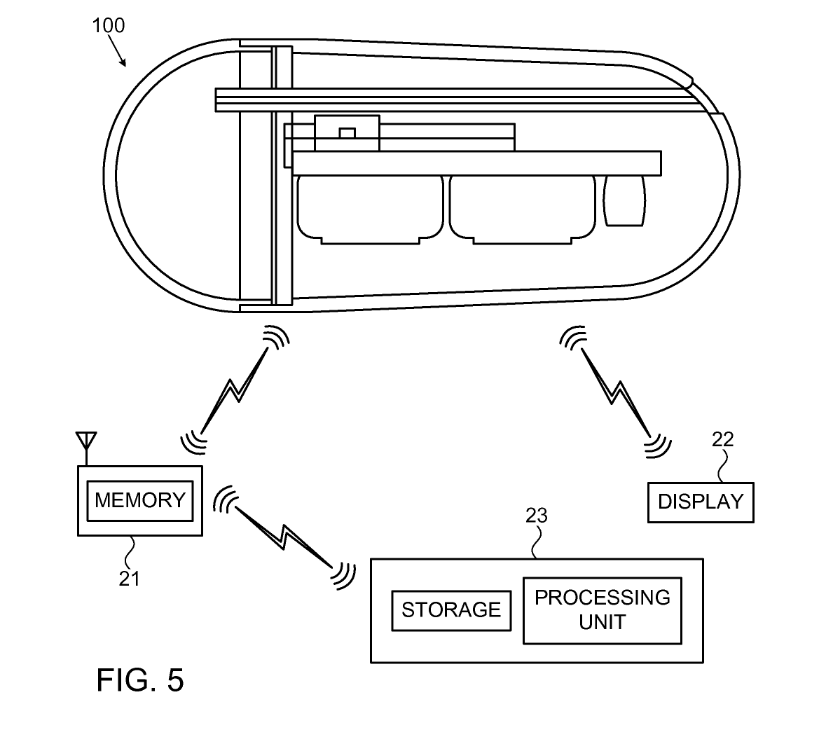

Reference is now made to FIG. 5, which presents a schematic illustration of a system for obtaining colorimetric or intensity data, or the like, from the in vivo diagnostic device. The system may include in vivo diagnostic device 100, an external receiver/recorder 21 able to receive data (e.g., image data) transmitted by in vivo device 100, computing platform (workstation) 23 able to store, process, and analyze the received data and optional display 22.

In a further embodiment, receiver 21 may comprise a memory unit for storing the data transmitted from in vivo device 100. The external receiver 21 may include one or more antenna elements or an antenna array, e.g., to improve signal reception and/or to allow localization of in-vivo device 100. Receiver 21 may possibly be close to or worn on a subject.

Receiver 21 may be operatively associated with a computing platform or workstation 23, which may, for example, store the received data (e.g., image data and/or other data), process the received data (e.g., using a processor), store the data in a storage unit, display the received data and/or processed data (e.g., using a monitor), analyze the data, perform post-processing operations, perform real-time processing operations, or the like.

In a particular embodiment, transmitter 10 may operate using radio waves; but in some embodiments, such as those where in vivo diagnostic device 100 is included within an endoscope, transmitter 10 may transmit/receive data via, for example, a wire, optical fiber and/or other suitable methods. Other known wireless methods of transmission may be used. Transmitter 10 may include, for example, a transmitter module or sub-unit and a receiver module or sub-unit, or an integrated transceiver or transmitter-receiver.

Transmitter 10 may also be capable of receiving signals/commands, for example from an external transceiver. For example, in one embodiment, transmitter 10 may include an ultra low power Radio Frequency (RF) high bandwidth transmitter, possibly provided in Chip Scale Package (CSP). In yet further embodiment, transmitter 10 may transmit/receive via an antenna (not shown).

In some embodiments, in vivo diagnostic device 100 may communicate with an external receiving and display system 22 (e.g., monitor) to provide display of data, control, or other functions. In some embodiments, display 22 may be a separate unit not part of computing device 23. Other embodiments may have other configurations and capabilities. For example, components may be distributed over multiple sites or units, and control information or other information may be received from an external source.

In some embodiments, display 22 may display the transmission spectra of the color signal from control line 16 and test line 17 appearing on strip 3 (as shown in FIG. 1). In other embodiments, display 22 may display the transmission spectra along with other information, e.g., pH values at the correlating in vivo locations of where the tested proteins are detected. In other embodiments, where the in vivo diagnostic device may for example comprise an imager and a broad band illumination, i.e., white light, in vivo images may be displayed either alone or alongside the in vivo locations along the GI tract where the body fluid sample is taken for in vivo immunoassay.

According to some embodiments, receiver 21 may be a disposable receiver. In some embodiments, receiver 21 may be a wearable disposable patch. A patient may wear receiver 21 and may swallow a new in vivo diagnostic device every day for a week, for example, in order to monitor the in vivo environment to detect the specific proteins. This is because the presence of specific proteins in the body fluid at specific locations along the GI tract may not always be constant, but rather the detected protein may appear one day, may disappear for a day or two, and may be noticed again on a different day. Therefore, there may be a need to monitor the GI tract during a long period of time, e.g., a week, by inserting to a patient a new device every day during an examination period.

Receiver 21 may include a visual indication which may show where along the GI tract the specific protein was detected. For example, receiver 21 may have several LEDs for every location along the GI tract, e.g., esophagus, stomach, small bowel and colon. The LEDs may light up when a detection of the specific protein is made by in vivo diagnostic device 100. For example, if calprotectin is detected in the small bowel, the LED corresponding to the small bowel may light up indicating to the patient and the physician of the patient's situation.

In a particular embodiment, a predetermined area of the pathology inside the GI tract, which should be diagnosed, may be labeled in advance, for example, by a color mark or an RFID tag implanted or fixed at or before said area, or by other methods. Such labeling may be carried out, for example, using a regular endoscope or maneuvered capsule endoscope. The in vivo diagnostic device of the invention may be equipped with a sensor to identify the mark, and the body lumen liquid in the identified area may be subjected to the in vivo immunoassay analysis. The sensor may be, for example, an imager or light-sensor and an image analysis unit capable of detecting a color mark or a scanner capable of detecting the proximity of the RFID tag. For example, if the sensor is an RFID scanner or any other sensor that is not based on detection of a color mark, the in vivo device may be free of the imaging components.

The in vivo diagnostic device of the invention may optionally include an in vivo camera/imager setup including one or more illumination sources and lenses (not shown) in order to acquire an in vivo image of a body lumen. These illumination sources may, for example, illuminate a body lumen or cavity being imaged and/or sensed. An optional optical system, including, for example, one or more optical elements, such as one or more lenses or composite lens assemblies, one or more suitable optical filters, or any other suitable optical elements, may optionally be included in the in vivo diagnostic device and may aid in focusing reflected light onto an imager (not shown), focusing illuminated light, and/or performing other light processing operations.

An in vivo diagnostic method according to embodiments of the invention is based on lateral flow immunoassay described above, which is performed on board of in vivo diagnostic device 100, as explained below.

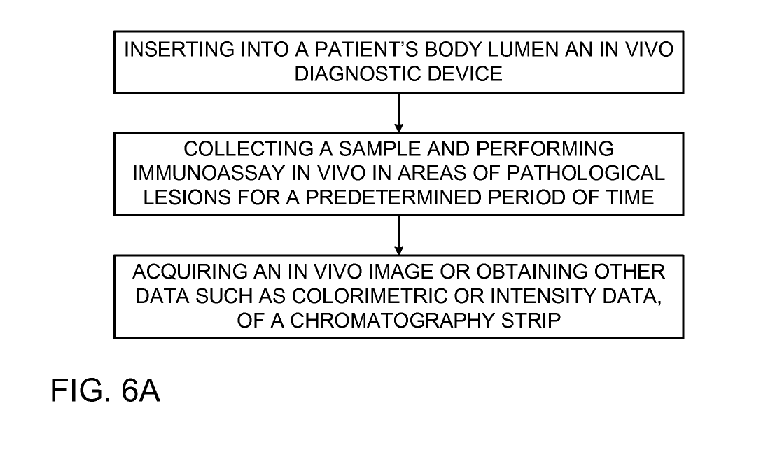

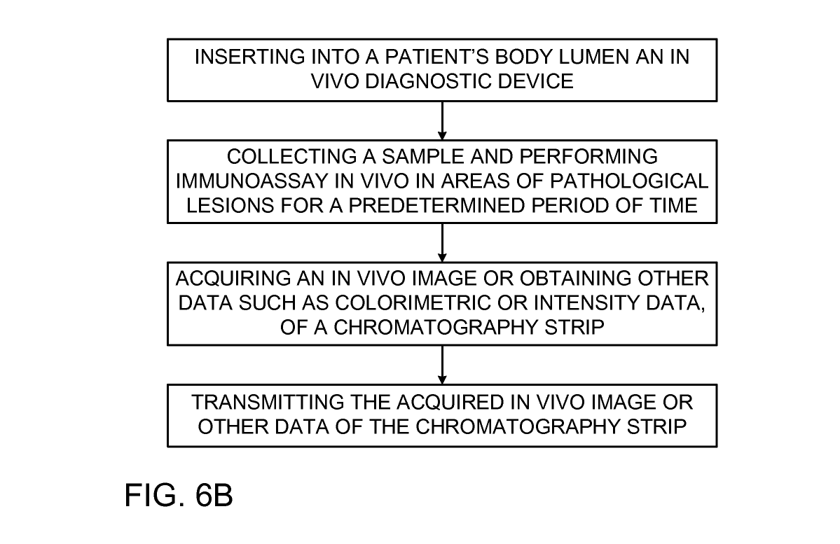

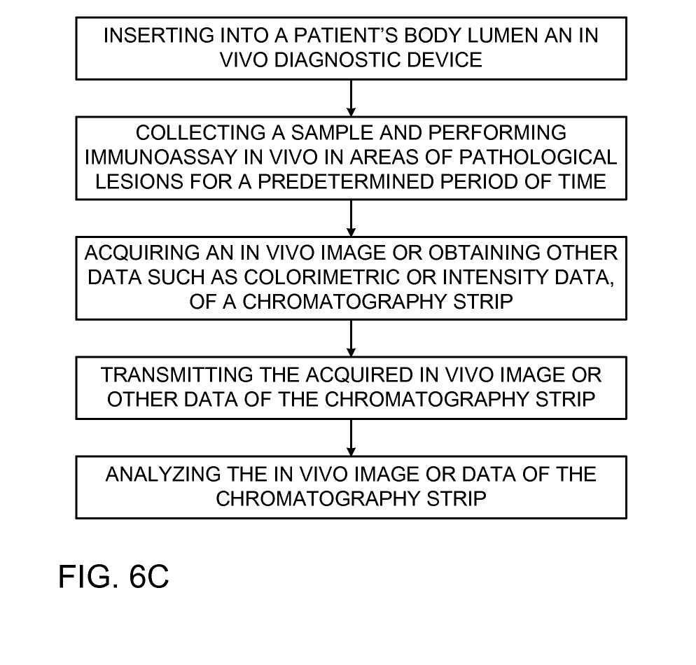

Reference is now made to FIGS. 6A-6C, which show flow charts describing methods of using an in vivo diagnostic device in accordance with embodiments of the present invention. According to FIG. 6A, the in vivo diagnostic method may comprise the following steps:
inserting into a patient's body lumen an in vivo diagnostic device according to embodiments of the invention;
collecting a sample and performing immunoassay in vivo in areas of pathological lesions for a predetermined period of time; and
acquiring an in vivo image or obtaining other data such as colorimetric or intensity data, of a chromatography strip.

According to FIG. 6B, a method may comprise the steps mentioned in FIG. 6A as well as a further step of transmitting the acquired in vivo image or other data of the chromatography strip to an external receiving device.

According to FIG. 6C, a method of using an in vivo diagnostic device in accordance with another embodiment of the invention, may comprise the steps mentioned in FIG. 6B as well as a further step of analyzing the in vivo image or data of the chromatography strip. The method in FIG. 6C may comprise other suitable operations.

Either of the methods illustrated in FIGS. 6A-6C may further optionally comprise acquiring in vivo an image of the body lumen.

In vivo diagnostic device 100 may be inserted into a body lumen, for example, into a patient's GI tract, e.g., by swallowing. Fluid samples from the body lumen may enter the device only through tube 2, and the samples may progress along chromatography strip 3 according to predetermined properties, for example, as known in the art of chromatography. Once, under predetermined conditions, sealing element 7 is opened, chromatography strip 3 located inside tube 2 comes in contact with endoluminal body fluids drawn into tube 2. The body fluids then advance along chromatography strip 3 due to the capillary action.

Along chromatography strip 3, the sample may initially react with a first reactant or substance at test line 16 (shown in FIG. 1), resulting in a visible marking on chromatography strip 3, and the resultant fluid may continue to progress along strip 3. A second reaction may occur at control line 17. Both lines are in view of sensor 6. Indications of both reactions, such as optical changes or images of areas of the lines 16 and 17 may be detected by sensor 6, and may be transmitted by transmitter 10 to an external receiving unit or recorder 21 outside a patient's body.

Generally, pH required for the immunoassay is around pH 7, although each particular immunoassay should be calibrated for its optimal range. In some embodiments, it may be essential to control the pH of the GI body fluid, especially of the stomach, taken for the in vivo analysis. Thus, prior to (or simultaneously with) inserting into a patient's body lumen an in vivo diagnostic device, a buffer reagent may be administered to the patient. This buffer reagent may be a combination of sodium bicarbonate, sodium citrate and potassium bicarbonate salts. An example of such buffers, which are called “antiacids” and clinically used for treatments of acidosis of the stomach and duodenum, heartburn, gastritis and ulcer, are Alkala N Powder (commercially available from SANUM-KEHLBECK GmbH & Co) and Alka Seltzer (commercially available from Bayer).

While most lateral flow immunoassays are only capable of providing a qualitative result, it is possible to obtain a certain degree of quantification by measuring the amount of conjugate bound to the capture zone. As mentioned above, this may be done by using a dedicated sensor 6 capable of measuring the intensity of the colored test line. By utilizing unique wavelengths of light for illumination in conjunction with either CMOS or CCD detection technology, a signal rich image can be produced of the actual test lines. Using image processing algorithms specifically designed by the common assignees of the present invention for a particular immunoassay type and medium, line intensities may then be correlated with analyte concentrations. More sophisticated techniques, such as fluorescent dye labeled conjugates, may also be implemented in order to improve the quantitative potential of the instant in vivo diagnostic method. Alternative non-optical techniques are also able to report quantitative assays results. One such example is a magnetic immunoassay in the lateral flow test form, which also allows getting a quantified result.

Receiver 21 connected to processor 23 (e.g., in a workstation or computing platform) may calculate a concentration or amount of the analyte in a sample, based on the data obtained from device 100. For example, colorimetric parameters or spectral parameters, such as intensity, hue, brightness, saturation, contrast, histogram data, or the like, may be used to calculate or determine the concentration or amount of the analyte.

In some embodiment, processor 23 may comprise, for example, a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit. In one embodiment, for example, the processing unit or controller may be embedded in or integrated with the transmitter, and may be implemented, for example, using an ASIC.

According to another embodiment, a processor on board the in-vivo diagnostic device may calculate or provide a quantitative determination of the color change in lines 16 and 17. This information may be transmitted to receiver 21 outside the patient's body. Such processing may be performed substantially in real-time or may be performed offline, e.g., using post processing operations.

In some embodiments, endoluminal body fluid drawn into in vivo diagnostic device 100 may include, for example, tumor markers. Tumor markers may include molecules occurring in body fluid or tissue that are associated with cancer. Typically, tumor markers may be cancerous cells or products of cancerous cells, and may represent aberrant production of what may be a typically normal element. Some markers, such as antibodies, may be produced in response to the presence of cancer. Tumor marker targeted molecules may have a high affinity to tumor markers and, under certain conditions, may adhere to tumor markers in a liquid environment. These may include antigens having specificity to tumor marker antibodies. Alternatively, tumor marker targeted molecules may include antibodies specific to tumor marker antigens. Body fluid samples may be analyzed for other chemicals, compounds or molecules.

Optionally, in vivo diagnostic device 100 may include one or more sensors, instead of or in addition to sensor 6. Other sensors may, for example, sense, detect, determine and/or measure one or more values of properties or characteristics of the surrounding of device 100. For example, device 100 may include a pH sensor, a temperature sensor, an electrical conductivity sensor, a pressure sensor, or any other known suitable in vivo sensor.

Various aspects of the various embodiments disclosed herein are combinable with the other embodiments disclosed herein. Although portions of the discussion herein may relate to chromatography “strips”, embodiments of the invention are not limited in this regard, and may include, for example, chromatography units, chromatography elements, chromatography components, chromatography testers, or the like, which may be strip-shaped, non strip-shaped, or may have various suitable shapes and dimensions.

Although portions of the discussion herein may relate to collection and/or release of fluid or body fluid, the present invention is not limited in this regard, and may include, for example, collection and/or release of one or more materials, substances, fluids, solids, gases, materials including both fluids and solids, or the like.

A device, system and method in accordance with some embodiments of the invention may be used, for example, in conjunction with a device which may be inserted into a human body. However, the scope of the present invention is not limited in this regard. For example, some embodiments of the invention may be used in conjunction with a device, which may be inserted into a non-human body or an animal body.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An in-vivo diagnostic device comprising:
a housing comprising:
a chamber;
a chromatography strip for immunoassay of a body lumen substance;
a casing for said chromatography strip, said casing having a first opening to allow entrance of in vivo liquids into the casing and a second opening into said chamber; and
a sensor to sense a property of the chromatography strip.

2. The device according to claim 1, wherein said device is an autonomous swallowable capsule.

3. The device according to claim 1, wherein said property of the chromatographic strip is selected from the following group: color, color intensity, color change, radiation, emitted signal, emitted radiation and a change of any of the aforementioned properties.

4. The device according to claim 1, wherein said sensor is selected from the following group: a photodiode, a photodiodes array, an electrochemical sensing unit, a magnetic field sensing unit, an imager, an image sensor, a light detector, a color detector, a light-sensitive unit and a color-sensitive unit.

5. The device according to claim 1, wherein said casing for the chromatography strip is in a form of a tube, a sleeve or a pipe.

6. The device according to claim 1, wherein said casing for the chromatography strip is transparent.

7. The device according to claim 1, wherein said device further comprises a sensor for sensing the current location of the device.

8. The device according to claim 1, wherein said device further comprises a transmitter and an antenna for transmitting data from the device to an external system.

9. The device according to claim 1, wherein said device further comprises an imager for acquiring an in vivo image of the chromatography strip.

10. The device according to claim 1, wherein said device further comprises a power source.

11. The device according to claim 1, wherein said device further comprises a camera for acquiring an in vivo image of a body lumen.

12. An in vivo diagnostic system comprising the in vivo diagnostic device of claim 1, an external receiver/recorder able to receive data transmitted by the in vivo device, and a computing platform or workstation able to store, process, display, or analyze the received data.

13. A method for in vivo diagnostics comprising the following steps:
inserting into a patient's body lumen an in vivo diagnostic device of claim 1;
collecting a sample and performing immunoassay in vivo in areas of pathological lesions for a predetermined period of time; and
acquiring an in vivo image or obtaining other data such as colorimetric or intensity data, of a chromatography strip.

14. The method according to claim 13, further comprising the steps of transmitting the acquired in vivo image or other data of the chromatography strip and analyzing the in vivo image or data of the chromatography strip.

15. The method according to claim 13, further comprising the step of acquiring in vivo an image of the body lumen.
